(19) United States
(12) Patent Application Publication
(10) Pub. No.: US 2025/0161410 A1
(43) Pub. Date: May 22, 2025

(54) COMPOSITION FOR PREVENTING OR TREATING MUSCLE DISEASE CONTAINING CXCL 14 AS AN ACTIVE INGREDIENT

(71) Applicant: SOONCHUNHYANG UNIVERSITY INDUSTRY ACADEMY COOPERATION FOUNDATION, Asan-si (KR)

(72) Inventor: Jeong-kyo YOON, Seongnam-si (KR)

(73) Assignee: SOONCHUNHYANG UNIVERSITY INDUSTRY ACADEMY COOPERATION FOUNDATION, Asan-si (KR)

(21) Appl. No.: 18/938,877

(22) Filed: Nov. 6, 2024

(30) Foreign Application Priority Data
Nov. 20, 2023 (KR) 10-2023-0160095

(51) Int. Cl. A61K 38/19; A61P 21/00

(52) U.S. Cl. CPC A61K 38/195; A61P 21/00

(57) ABSTRACT

The present disclosure relates to a composition for preventing or treating muscle disease, comprising CXCL14 as an active ingredient. According to the present disclosure, it was confirmed that CXCL14 increased muscle differentiation and synthesis, thereby increasing muscle mass. In addition, it was confirmed that in LPS-induced muscular atrophy or DEX-induced muscular dystrophy, CXCL14 inhibited muscle loss, increased the expression of muscle synthesis and differentiation factors, and inhibited the expression of muscle degradation proteins. In addition, it was confirmed that CXCL14 promoted muscle synthesis and differentiation and inhibited the muscle degradation even in vivo.

### CROSS-REFERENCE TO RELATED APPLICATIONS

This application claims priority to Korean Patent Application No. 10-2023-0160095 filed Nov. 20, 2023, the entire disclosure of which is incorporated herein by reference.

### INCORPORATION BY REFERENCE OF SEQUENCE LISTING

The instant application contains a Sequence Listing which has been filed electronically in xml format and is hereby incorporated by reference in its entirety. Said xml copy, created on Nov. 6, 2024, is named Q303687_sequence listing as filed.xml and is 10,501 bytes in size.

### TECHNICAL FIELD

The present disclosure relates to a composition for preventing or treating muscle disease comprising CXCL14 as an active ingredient.

### BACKGROUND

Muscle accounts for approximately 40% of the human body, and is essentially required to secure an appropriate amount of muscle to maintain the functional ability of the human body and prevent metabolic diseases. The muscle is broadly divided into smooth muscle, cardiac muscle, and skeletal muscle, and the skeletal muscle accounts for a significant portion of the entire body and facilitates the movement of the skeleton. The skeletal muscle is an organ that occupies the largest part of the human body and accounts for 40% to 50% of the total body weight, and plays an important role in various metabolic functions in the body, including energy homeostasis and heat generation. Human muscles decrease by 1% or more per year after the age of 40, and by the age of 80, approximately 50% of maximum muscle mass is lost, and muscle loss in the old age is recognized as the most important factor in reducing an overall physical function.

The types of muscle fibers constituting the muscle are mainly classified into Type I, Type IIA, and Type IIB according to a metabolic process that generates ATP and a speed of contraction. ‘Type I muscle fiber’ has a slow contraction speed and contains a large number of myoglobin and mitochondria to be suitable for continuous and low-intensity aerobic activity. Type I muscle fiber is red in color and also called red muscle, and soleus is a representative example of this type. On the other hand, ‘Type IIB muscle fiber’ is used for very short, high-intensity anaerobic exercise due to a fast contraction speed, and has a low myoglobin content to be white in color. ‘Type IIA muscle fiber’ has intermediate characteristics between the two types of muscle fiber mentioned above. With age, not only a composition of Type I and II muscle fibers varies for each muscle area, but also all types of muscle fibers also decrease.

The skeletal muscle has the characteristic of being regenerated and maintained depending on an environment, but such a characteristic is lost with age, and as a result, as aging progresses, not only muscle mass decrease, but muscle strength is also lost.

Sarcopenia is a condition in which the amount and function of skeletal muscle are reduced. The sarcopenia is caused by various factors such as aging, hormonal disorders, undernutrition, lack of physical activity, inflammation, and degenerative diseases, but among them, cancer, aging, and lack of sex hormones are known to be the main causes. As average life expectancy increases worldwide due to advances in medical technology and the development of various therapeutic agents, the aging population is increasing, and accordingly, the demand for treatment of sarcopenia is also expected to continue to increase. In patients with sarcopenia, the number of myoblasts decreases due to impairment in the recruitment, activation, or proliferation of satellite cells, which are stem cells of myoblasts, and the proliferation and differentiation of myoblasts decrease, and accordingly, the size and number of muscle fibers decrease at the histological level in the muscles of patients with sarcopenia, resulting in symptoms of decreased muscle function. Over the past decade, as a study on the epidemiology of sarcopenia has been actively conducted mainly in the United States and Europe, an interest in the clinical importance of sarcopenia has recently exploded. Although early studies have mainly shown results that sarcopenia causes a decrease in quality of life due to systemic weakness, activity impairment, and decreased muscle strength, recently published studies have reported that in addition to quality of life, the risk of osteoporotic fractures may significantly increase. In addition, sarcopenia is receiving attention as a disease that needs to be treated appropriately because chronic diseases such as diabetes, metabolic syndrome, obesity, chronic renal failure, and chronic liver failure are caused in patients with sarcopenia, and ultimately, mortality is also increased. Recently, in the United States, it has been reported that in patients with sarcopenia, the possibility to develop a physical disability is approximately 1.5 times to 3.5 times increased to cause annual social cost of USD 18.5 billion per year. According to the National Health and Nutrition Survey in Korea, the prevalence of sarcopenia is 42.0% for men and 42.7% for women aged 60 or older, which is a very common disease. In particular, due to the highest rate of aging even in the world, in Korea, it is certain to become an important social problem in the future.

### SUMMARY

The present disclosure is directed to provide a pharmaceutical composition for preventing or treating muscle disease, including a CXC motif chemokine ligand 14 (CXCL14) protein as an active ingredient.

The present disclosure is also directed to provide a food composition for preventing or improving muscle disease, including a CXC motif chemokine ligand 14 (CXCL14) protein as an active ingredient.

In order to achieve the above aspects, the present disclosure provides a pharmaceutical composition for preventing or treating muscle disease, including a CXC motif chemokine ligand 14 (CXCL14) protein as an active ingredient.

The present disclosure also provides a food composition for preventing or improving muscle disease, including a CXC motif chemokine ligand 14 (CXCL14) protein as an active ingredient.

According to the present disclosure, it was confirmed that CXCL14 increased muscle mass by increasing muscle differentiation and synthesis. In addition, it was confirmed that in LPS-induced muscular atrophy or DEX-induced muscular atrophy, CXCL14 inhibited muscle loss, increased the expression of muscle synthesis and differentiation factors, and inhibited the expression of muscle degradation proteins. In addition, CXCL14 promotes muscle synthesis and differentiation and inhibits muscle degradation even in vivo, and thus may be usefully utilized in related industries.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a diagram showing the expression of myosin heavy chains and muscle mass of myofiber cells according to CXCL14 treatment of the present disclosure (A: immunofluorescence staining result, B: muscle mass quantification).

FIG. 2 is a diagram showing Western blot analysis of the expression of muscle synthesis, differentiation, and degradation factors of myofiber cells according to CXCL14 treatment of the present disclosure.
A: Confirmation of AKT-S6K pathway up to 2 hours of CXCL14 treatment
B: Confirmation of expression of FOXO1/3 and muscle degradation factors up to 2 hours of CXCL14 treatment
C: Confirmation of expression of muscle synthesis, differentiation, and degradation factors 48 hours after CXCL14 treatment

FIG. 3 is a diagram showing a muscular atrophy inhibitory effect according to CXCL14 treatment in myofiber cells with muscular atrophy induced by LPS (A: immunofluorescence staining result, B: muscle mass quantification).

FIG. 4 is a diagram showing Western blot analysis of the expression of muscle synthesis, differentiation, and degradation factors according to CXCL14 treatment in myofiber cells with muscular atrophy induced by LPS (A: confirmation of AKT-S6K pathway, B: confirmation of FOXO1/3 and muscle degradation factors).

FIG. 5 is a diagram showing a muscular atrophy inhibitory effect according to CXCL14 treatment in myofiber cells with muscular atrophy induced by dexamethasone (A: immunofluorescence staining result, B: muscle mass quantification).

FIG. 6 is a diagram showing Western blot analysis of the expression of muscle synthesis, differentiation, and degradation factors according to CXCL14 treatment in myofiber cells with muscular atrophy induced by dexamethasone (A: confirmation of AKT-S6K pathway, B: confirmation of FOXO1/3 and muscle degradation factors).

FIG. 7 is a schematic diagram of an experiment to confirm a muscle increasing effect in vivo according to CXCL14 treatment of the present disclosure.

FIG. 8 is a diagram confirming a muscle increasing effect according to the local injection of a CXCL14 expression vector of the present disclosure by immunofluorescence staining and cross sectional area (CSA) index (A: immunofluorescence staining result, B: CSA index quantification, C: CSA median quantification).

FIG. 9 is a diagram showing Western blotting analysis of an AKT-S6K pathway and the expression of muscle degradation factors according to the local injection of the CXCL14 expression vector of the present disclosure.

FIG. 10 is a schematic diagram of an experiment to confirm an LPS-induced muscular atrophy improvement effect according to the local injection of the CXCL14 expression vector of the present disclosure.

FIG. 11 is a diagram confirming a muscular atrophy inhibitory effect according to the local injection of a CXCL14 expression vector of the present disclosure in an LPS-induced muscular atrophy animal model by immunofluorescence staining and cross sectional area (CSA) index (A: immunofluorescence staining result, B: CSA index quantification, C: CSA median quantification).

FIG. 12 is a diagram showing Western blot analysis of the expression of FOXO1/3 and muscle degradation factors according to the local injection of a CXCL14 expression vector in a LPS-induced muscular atrophy animal model.

FIG. 13 is a schematic diagram of an experiment to confirm a dexamethasone-induced muscular atrophy improvement effect according to the local injection of the CXCL14 expression vector of the present disclosure.

FIG. 14 is a diagram confirming a muscular atrophy inhibitory effect according to the local injection of a CXCL14 expression vector in a dexamethasone-induced muscular atrophy animal model by immunofluorescence staining and cross sectional area (CSA) index (A: immunofluorescence staining result, B: CSA index quantification, C: CSA median quantification).

FIG. 15 is a diagram showing Western blot analysis of the expression of FOXO1/3 and muscle degradation factors according to the local injection of a CXCL14 expression vector in a dexamethasone-induced muscular atrophy animal model.

### DETAILED DESCRIPTION

Hereinafter, exemplary embodiments of the present disclosure will be described in detail with reference to the accompanying drawings. In the following description, detailed descriptions of techniques well-known to those skilled in the art may be omitted. Further, in describing the present disclosure, the detailed description of associated known functions or constitutions will be omitted if it is determined to unnecessarily make the gist of the present disclosure unclear. In addition, terminologies used in the present disclosure are terminologies used to properly express preferred exemplary embodiments of the present disclosure, which may vary according to a user, an operator's intention, or customs in the art to which the present disclosure pertains.

Accordingly, definitions of the terminologies need to be described based on contents throughout this specification. Throughout this specification, unless explicitly described to the contrary, when a certain part “comprises” a certain component, it will be understood to imply the inclusion of stated elements, not the exclusion of any other elements.

The present disclosure provides a pharmaceutical composition for preventing or treating muscle disease, including a CXC motif chemokine ligand 14 (CXCL14) protein as an active ingredient.

As used in the present disclosure, the term “prevention” refers to any action that suppresses the symptoms of a specific disease or delays its progression by administering the composition of the present disclosure.

As used in the present disclosure, the term “treatment” refers to any action that improves or beneficially changes the symptoms of a specific disease by administering the composition of the present disclosure.

The pharmaceutical composition of the present disclosure may further include an adjuvant in addition to the active ingredient. The adjuvant may be used with any adjuvant known in the art without limitation, but further include, for example, a Freund's complete adjuvant or an incomplete adjuvant to increase the effect thereof.

The pharmaceutical composition according to the present disclosure may be prepared in the form of incorporating the active ingredient into a pharmaceutically acceptable carrier. Here, the pharmaceutically acceptable carrier includes carriers, excipients and diluents commonly used in the pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present disclosure may be formulated and used in the form of oral formulations, such as powders, granules, tablets, capsules, suspensions, emulsions, syrups, and aerosols, external preparations, suppositories, and sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one or more excipients, for example, starch, calcium carbonate, sucrose, lactose, gelatin, etc. with the active ingredient. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used diluents, such as water and liquid paraffin. Formulations for parenteral administration include sterile aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, etc. may be used. As the base material of the suppository, witepsol, Tween 61, cacao butter, laurinum, glycerogelatin, etc. may be used.

The pharmaceutical composition according to the present disclosure may be administered to a subject through various routes. All methods of administration may be expected, and the pharmaceutical composition may be administered by, for example, oral, intravenous, intramuscular, subcutaneous, and intraperitoneal injection.

The dose of the pharmaceutical composition according to the present disclosure is selected in consideration of the age, body weight, sex, physical conditions, and the like of a subject. It is obvious that the concentration of the active ingredient included in the pharmaceutical composition may be variously selected according to a subject, and preferably included in the pharmaceutical composition at a concentration of 0.01 to 5,000 μg/ml. When the concentration is less than 0.01 μg/ml, pharmaceutical activity may not be exhibited, and when the concentration exceeds 5,000 μg/ml, toxicity to the human body may be exhibited.

In addition, the CXCL14 protein included in the pharmaceutical composition of the present disclosure includes a protein having a substantially equivalent physiological activity to the protein. The CXCL14 protein having the substantially equivalent physiological activity includes functional equivalents and functional derivatives to the protein. The “functional equivalent” refers to an amino acid sequence variant in which some or all of amino acids of a native protein are substituted, or some of the amino acids are deleted or added, and has a substantially equivalent physiological activity to the native CXCL14 protein. The “functional derivative” refers to a protein that has been modified to increase or decrease the physicochemical properties of the CXCL14 protein and has a substantially equivalent physiological activity to the native CXCL14 protein.

According to an exemplary embodiment of the present disclosure, the CXCL14 protein may include an amino acid sequence represented by SEQ ID NO: 1.

According to an exemplary embodiment of the present disclosure, the composition may further include a CXCL14 expression vector, and the CXCL14 expression vector may be a plasmid including a base sequence represented by SEQ ID NO: 2 or a base sequence represented by SEQ ID NO: 3.

A plasmid expression vector is a method of directly delivering plasmid DNA to human cells as a gene transfer method approved by the FDA capable of being used in humans (Nabel, E. G., et al., Science, 249:1285-1288, 1990). Unlike viral vectors, the plasmid DNA has an advantage of being able to be homogeneously purified. As the plasmid expression vector that may be used in the present disclosure, a mammalian expression plasmid known in the art may be used. Representative examples include, but are not limited to, pRK5 (EP U.S. Pat. No. 307,247), pSV16B (WO Patent No. 91/08291), pVL1392 (PharMingen), and the like.

The plasmid expression vector including the polynucleotide according to the present disclosure may be introduced into cells by methods known in the art, but are not limited to, for example, transient transfection, microinjection, transduction, cell fusion, calcium phosphate precipitation, liposome-mediated transfection, DEAE dextran-mediated transfection, polybrene-mediated transfection, electroporation, gene gun, and other known methods for introducing DNA into cells (Wu et al., J. Bio. Chem., 267:963-967, 1992; Wu and Wu, J. Bio. Chem., 263:14621-14624, 1988).

The vector capable of expressing the CXCL14 may be administered by a known method. For example, the vector may be administered topically, parenterally, orally, nasally, intravenously, intramuscularly, subcutaneously, or by other appropriate means.

According to an exemplary embodiment of the present disclosure, the CXCL14 may increase muscle mass, and the increasing of the muscle mass may be increasing the number of myosin heavy chain positive cells.

According to an exemplary embodiment of the present disclosure, the CXCL14 may increase the expression of muscle synthesis and differentiation factors, and the muscle synthesis and differentiation factors may be AKT (protein kinase B)-S6K (Ribosomal protein S6 kinase) pathway factors, and the AKT-S6K pathway factor may be selected from the group consisting of p-ATK (T308), p-AKT (S473), total AKT, p-S6K (T389), total S6K, p-mTOR (Mammalian target of rapamycin), and total mTOR.

According to an exemplary embodiment of the present disclosure, the muscle synthesis and differentiation factors may be Forkhead box protein 01 or Forkhead box protein 03 (FOXO3).

According to an exemplary embodiment of the present disclosure, the CXCL14 may inhibit the expression of a muscle degradation factor, and the muscle degradation factor may be Atrogin-1 or Muscle RING-finger protein-1 (MuRF1).

According to an exemplary embodiment of the present disclosure, the muscle disease may be disease selected from the group consisting of muscular atrophy, myopathy, muscular degeneration, myasthenia, muscular injury, dystrophinopathy, myopathy, muscular dystrophy, cachexia, and sarcopenia, and preferably sarcopenia or muscular atrophy, but is not limited thereto.

According to an exemplary embodiment of the present disclosure, the muscle disease may be induced by bacterial infection or steroid.

The present disclosure also provides a food composition for preventing or improving muscle disease, including a CXC motif chemokine ligand 14 (CXCL14) protein as an active ingredient.

As used in the present disclosure, the “improvement” means all actions that at least reduce parameters associated with conditions to be treated, such as the severity of symptoms.

In addition to containing the active ingredient of the present disclosure, the food composition of the present disclosure may contain various flavoring agents, natural carbohydrates, or the like as an additional ingredient, like conventional food compositions.

Examples of the above-described natural carbohydrates include conventional sugars, including monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. The above-described flavoring agents may be advantageously used with natural flavoring agents (thaumatin), stevia extracts (e.g., rebaudioside A, glycyrrhizin, etc.), and synthetic flavoring agents (saccharin, aspartame, etc.). The food composition of the present disclosure may be formulated in the same manner as the pharmaceutical composition to be used as a functional food or added to various foods. The foods capable of adding the composition of the present disclosure include, for example, beverages, meat, chocolate, foods, confectionery, pizza, ramen, other noodles, gums, candies, ice creams, alcohol beverages, vitamin complexes, health food supplements, etc.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like, in addition to the extract as the active ingredient. In addition, the food composition of the present disclosure may contain pulps for preparing natural fruit juice, fruit juice beverages, and vegetable beverages.

The functional food composition of the present disclosure may be prepared and processed in the form of tablets, capsules, powders, granules, liquids, pills, etc. for the purpose of prevention or treatment of muscle disease. In the present disclosure, the ‘health functional food composition’ refers to foods prepared and processed by using raw materials or ingredients with functionality, which are useful for the human body according to the Art No. 6727 on Health Functional Food, and means foods taken for adjusting nutrients for the structures and functions of the human body or obtaining a useful effect on health applications such as physiological actions. The health functional food of the present disclosure may include conventional food additives, and the suitability as the food additives is determined by the specifications and standards for the corresponding item in accordance with the general rules of the Food Additives Codex, general test methods, etc., that are approved by the Food and Drug Administration, unless otherwise specified. The items disclosed in the ‘Food Additives Codex’ may include, for example, chemical composites such as ketones, glycine, calcium citrate, nicotinic acid, and cinnamic acid; natural additives such as persimmon color, licorice extract, crystal cellulose, Kaoliang color, and guar gum; mixed formulations such as sodium L-glutamic acid formulations, noodle additive alkali agents, preservative formulations, and tar color formulations. For example, the health functional food in the form of tablets may be prepared by granulating a mixture obtained by mixing the active ingredient of the present disclosure with an excipient, a binder, a disintegrant, and other additives using a conventional method, and then compression-molding the mixture by adding a slip modifier and the like, or directly compression-molding the mixture. In addition, the health functional food in the form of tablets may also contain a flavors enhancer or the like as needed. In the health functional food in the form of capsules, hard capsules may be prepared by filling a mixture mixed with the active ingredient of the present disclosure and additives such as excipients into conventional hard capsules, and soft capsules may be prepared by filling a mixture mixed with the active ingredient of the present disclosure and additives such as excipients into capsule bases such as gelatin. The soft capsules may contain a plasticizer such as glycerin or sorbitol, a colorant, a preservative, and the like, if necessary. The health functional food in the form of pills may be prepared by molding a mixture obtained by mixing the active ingredient of the present disclosure with an excipient, a binder, a disintegrant, etc. by conventional known methods, and may also be coated with white sugar or other coating agents or surface-coated with materials such as starch and talc, if necessary. The health functional food in the form of granules may be prepared by granulizing a mixture obtained by mixing the active ingredient of the present disclosure with an excipient, a binder, a disintegrant, etc. by conventional known methods and may contain a flavoring agent, a flavors enhancer, etc., if necessary.

Hereinafter, the present disclosure will be described in more detail through Examples. These Examples are to explain the present disclosure in more detail, and it will be apparent to those skilled in the art that the scope of the present disclosure is not limited to these Examples.

### <Example 1> Confirmation of Increased Myofiber Cell Mass by CXCL14

The effect of increasing myofiber cell mass by CXCL14, a novel protein of the present disclosure, was confirmed. Specifically, 1×10⁵ C2C12 cells as a mouse muscle myoblast cell line were inoculated into each well of a collagen-coated 12-well plate using a DMEM/FBS10% culture medium, and then cultured until the cells became confluent (24 hours). The cell culture medium was replaced with a DMEM/2% horse serum culture medium, and then further cultured for 4 days to obtain myofiber cells through induced muscle differentiation. The culture medium of the obtained myofiber cells was replaced again with DMEM/FBS10% to prevent further muscle differentiation, and then treated with a CXC motif chemokine ligand 14 (CXCL14) recombinant protein at a concentration of 20 or 100 ng/ml, and then cultured for 48 hours. After the culture was completed, the cells were fixed and the expression of a myosin heavy chain protein was confirmed using immunofluorescence staining. As a control group, a negative control group treated with the same amount of PBS was used, and an amino acid sequence (SEQ ID NO: 1) of the recombinant CXCL14 protein of the present disclosure was shown in Table 1 below.

TABLE 1

| Name | Sequence |
|---|---|
| CXCL14 amino acid | SKCKCSRKGPKIRYSDVKKLEMKPKYP |
| sequence (N—C) | HCEEKMVIITTKSVSRYRGQEHCLHPK |
| | LQSTKRFIKWYNAWNEKRRVYEE |

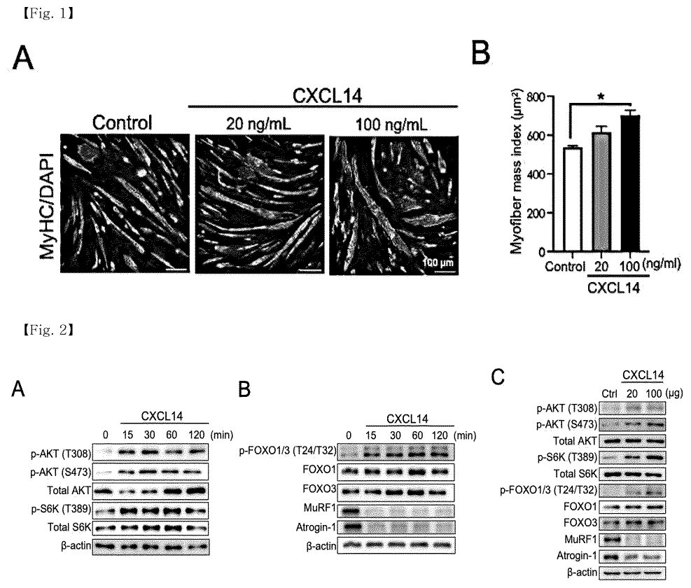

As a result, as shown in FIG. 1, it was confirmed that compared to the control group, in a group treated with CXCL14, the thickness of the myofiber cell was increased in a concentration-dependent manner (FIG. 1A), and as a quantitative value of the fluorescence expression of myofiber cells, CXCL14 increased the mass index of myofiber cells (FIG. 1B).

### <Example 2> Confirmation of Regulation of Expression of Myofiber Cell Synthesis and Differentiation Factors by CXCL14

The regulation of expression of myofiber cell synthesis and differentiation factors by CXCL14, a recombinant protein of the present disclosure, was confirmed. Specifically, C2C12 cells were cultured in the same manner as in Example 1, and then the expression of myofiber cell synthesis and differentiation factors up to 2 hours after CXCL14 treatment and the expression of factors 48 hours after treatment were analyzed by Western blotting. As the myofiber cell synthesis factor, Total AKT (protein kinase B) and AKT activation of an AKT-S6K pathway were confirmed by phosphorylation of T308 (threonine 308) and S473 (serine 473), and confirmed by phosphorylation of Total S6K (Ribosomal protein S6 kinase) and S6K (T389). In addition, the expression of Forkhead box protein 01 (FOXO1) and Forkhead box protein 03 (FOXO3), which were protein degradation regulators, was confirmed, and the expression of Muscle RING-finger protein-1 (MuRF1) and Atrogin-1 (known as F-box only protein 32, FBXO32), which were muscle protein degradation enzymes, was analyzed, respectively.

As a result, as shown in FIG. 2, it was confirmed that compared to the control group, in the group treated with CXCL14 (100 ng/ml), the activity of AKT-S6K was increased in a time-dependent manner up to 2 hours after CXCL14 treatment, and the phosphorylation of FOXO1/3 as the protein degradation regulator was increased. In addition, it was confirmed that the expression of the muscle protein degradation enzymes MuRF1 and Atrogin-1 was significantly reduced (FIGS. 2A and 2B). In addition, 48 hours after CXCL14 treatment, the activity of AKT-S6K and the phosphorylation of FOXO1/3 were increased in a concentration-dependent manner, thereby increasing the activity of FOXO1 and FOXO3, and the expression of MuRF1 and Atrogin-1 also decreased in a concentration-dependent manner (FIG. 2C). It was confirmed that the CXCL14 recombinant protein induced an increase in the mass of myofiber cells by increasing protein synthesis and inhibiting protein degradation in the myofiber cells.

### <Example 3> Confirmation of the Inhibitory Effect of CXCL14 on LPS-Induced Muscular Atrophy

In order to confirm the effect of CXCL14 of the present disclosure on improving muscular atrophy caused by bacterial infection, a Lipopolysaccharide (LPS)-induced muscular atrophy inhibitory effect was confirmed. Specifically, C2C12 cells were cultured in the same manner as in Example 1, and then treated with LPS at a concentration of 100 ng/ml, and treated with a CXCL14 recombinant protein at 100 ng/ml, and then cultured for 48 hours. After the culture was completed, the expression of a myosin heavy chain protein was analyzed by immunofluorescence staining, and the expression of muscle synthesis and differentiation factors of Example 2 and Total mTOR was analyzed by Western blotting. As a control group, an untreated control group (Control), an LPS group in which muscular atrophy was induced by LPS, and a group treated only with CXCL14 were used.

As a result, as shown in FIG. 3, it was confirmed that compared to the control group, in the LPS group, the expression of the myosin heavy chain was significantly decreased, but the expression of the myosin heavy chain decreased by LPS was significantly increased by CXCL14 treatment.

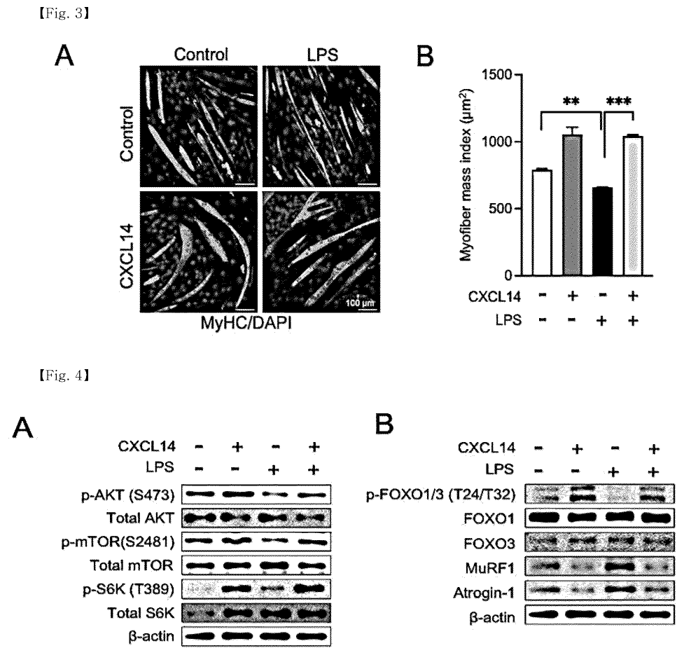

In addition, as shown in FIG. 4, as a result of confirming the expression of muscle synthesis and differentiation factors, it was confirmed that compared to the control group, in the LPS-treated group, the activity of AKT-S6K-related factors, FOXO1 and FOXO3 was significantly decreased, but CXCL14 significantly increased the activity of AKT-S6K-related factors, FOXO1 and FOXO3 decreased by LPS. In addition, it was confirmed that the expression of muscle protein degradation enzymes MuRF1 and Atrogin-1 was increased by LPS treatment, but the increased protein degradation enzymes were significantly decreased by CXCL14 treatment, so that CXCL14 improved muscular atrophy induced by bacterial infection.

### <Example 4> Confirmation of the Inhibitory Effect of CXCL14 on Dexamethasone-Induced Muscular Atrophy

In order to confirm the effect of CXCL14 of the present disclosure on improving steroid-induced muscular atrophy, a dexamethasone (DEX)-induced muscular atrophy inhibitory effect was confirmed. Specifically, C2C12 cells were cultured in the same manner as in Example 1, and then treated with dexamethasone at a concentration of 10 μM and treated with a CXCL14 recombinant protein at 100 ng/ml, and then cultured for 48 hours. After the culture was completed, the expression of a myosin heavy chain protein was analyzed by immunofluorescence staining, and the expression of muscle synthesis and differentiation factors of Example 2 and Total mTOR was analyzed by Western blotting. As a control group, an untreated control group (Control), a DEX group in which muscular atrophy was induced with dexamethasone, and a group treated only with CXCL14 were used.

As a result, as shown in FIG. 5, it was confirmed that compared to the control group, in the DEX group, the expression of a myosin heavy chain was significantly decreased, but the expression of the myosin heavy chain decreased by DEX was significantly increased by CXCL14 treatment.

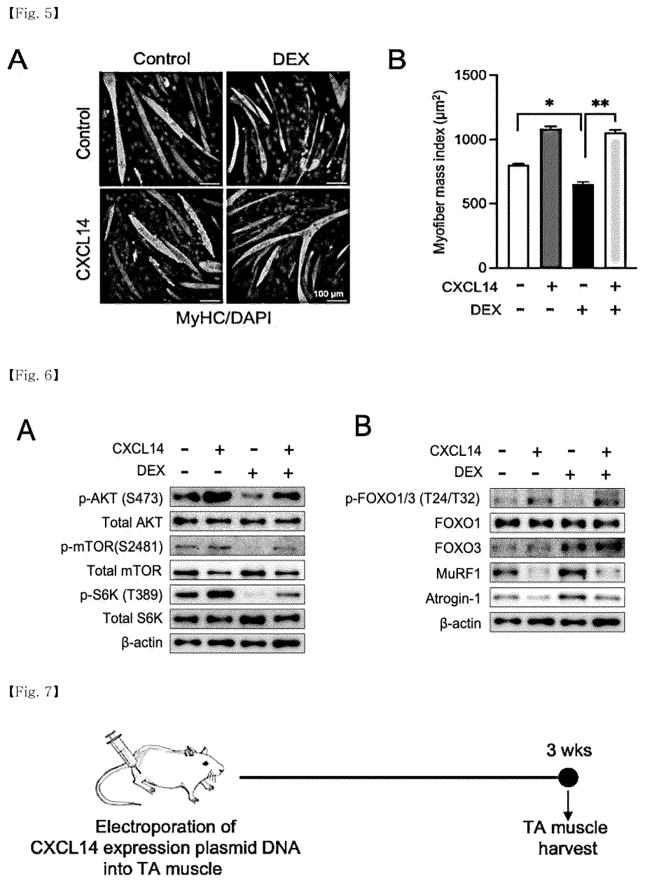

In addition, as shown in FIG. 6, as a result of confirming the expression of muscle synthesis and differentiation factors, it was confirmed that compared to the control group, in the DEX-treated group, the activity of AKT-S6K-related factors, FOXO1 and FOXO3 was significantly decreased, but CXCL14 significantly increased the activity of AKT-S6K-related factors, FOXO1 and FOXO3 decreased by DEX. In addition, it was confirmed that the expression of muscle protein degradation enzymes MuRF1 and Atrogin-1 was increased by DEX treatment, but the increased protein degradation enzymes were significantly decreased by CXCL14 treatment, so that CXCL14 improved steroid-induced muscular atrophy.

### <Example 5> Confirmation of In Vivo Muscle Increase by Local Expression of CXCL14

It was confirmed whether local expression of CXCL14 of the present disclosure increased muscle mass. Specifically, two types of human CXCL14 gene expression plasmid DNA (CXCL14-Myc; SEQ ID NO: 2 or HA-CXCL14; SEQ ID NO: 3) were each injected into the tibialis anterior muscle (TA) of C57BL/6 mice by electroporation, and stabilized for 3 weeks. Thereafter, the mice were humanely sacrificed, the tibialis anterior muscle was extracted, and muscle transverse paraffin sections were prepared. The control group was used as a control group injected with an empty vector, Mock plasmid, and the overall experimental process was shown in FIG. 7. Thereafter, CXCL14 protein expression was confirmed together with laminin protein expression by immunofluorescence staining using an anti-Myc antibody for CXCL14-Myc with Myc-epitope tag at the C-terminus, and an anti-HA antibody for HA-CXCL14 with HA (Hemagglutinin)-epitope tag at the N-terminus. In addition, the cross-sectional area (CSA) of a myofiber cell was measured using ImageJ program, which was used as a measure of myofiber cell mass.

In addition, Western blot analysis was used to measure the expression and activation levels of key factors in protein synthesis and degradation processes in muscle tissue lysates.

As a result, as shown in FIG. 8, it was confirmed that CXCL14 was located in the interstitial space between the myofiber cells, and a Laminin protein was expressed in the basal lamina around the myofiber cells. In addition, it was confirmed that in the group injected with CXCL14 plasmid DNA, compared to the control group, the CSA value increased, and the CSA median value significantly increased (FIGS. 8B and 8C).

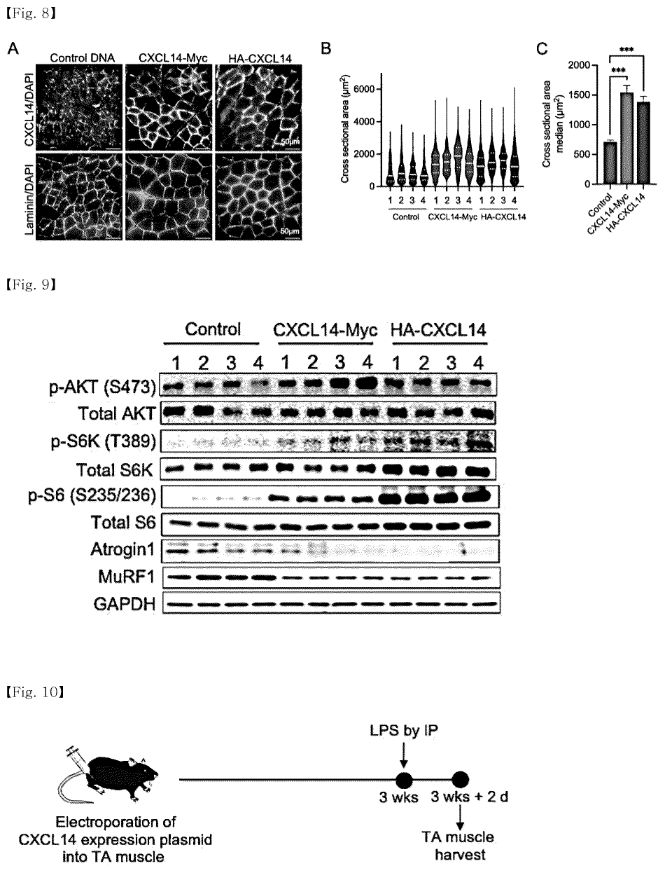

In addition, as shown in FIG. 9, it was confirmed that the expression of CXCL14 increased the activity of AKT-S6K signaling. In addition, it was confirmed that the expression of the muscle protein degradation enzymes Atrogin-1 and MuRF1 was decreased.

### <Example 6> Confirmation of Inhibition of LPS-Induced Muscular Atrophy by Local Expression of CXCL14

It was confirmed whether the local expression of CXCL14 of the present disclosure inhibited LPS-induced muscular atrophy in vivo. Specifically, a CXCL14 gene expression plasmid (20 μg) was injected into the TA muscle of C57BL/6 mice by electroporation, and then stabilized for 3 weeks. Thereafter, LPS (1 mg/kg of body weight) was injected intraperitoneally once to induce muscular atrophy. Two days after LPS injection, the mice were humanely sacrificed, TA muscles were extracted, and muscle transverse paraffin sections were prepared. The overall experimental process was shown in FIG. 10. Thereafter, the expression of CXCL14 protein and laminin protein was confirmed by immunofluorescence staining. In addition, the cross-sectional area (CSA) of a myofiber cell was measured using ImageJ program, which was used as a measure of myofiber cell mass.

In addition, Western blot analysis was used to measure the expression and activation levels of key factors in protein synthesis and degradation processes in muscle tissue lysates.

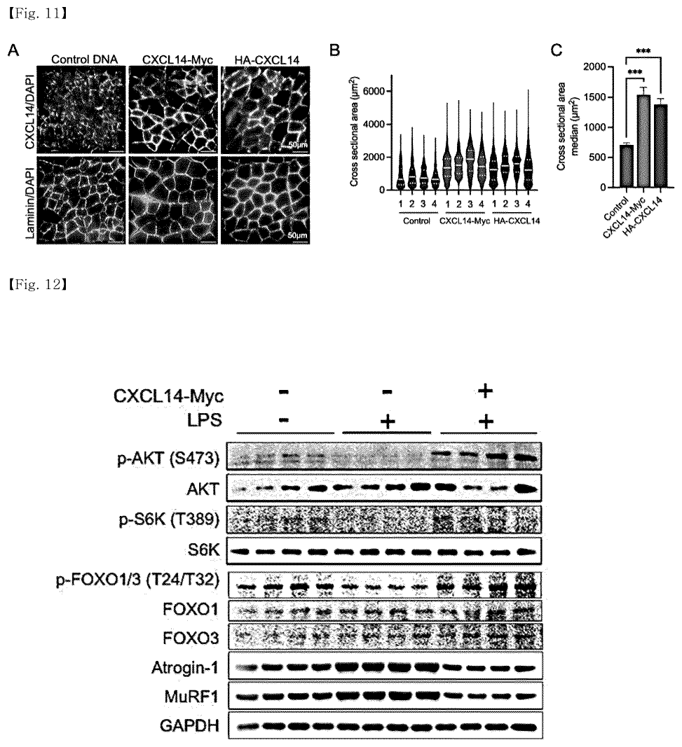

As a result, as shown in FIG. 11, it was confirmed that compared to the control group, in the LPS-injected group, the CSA value and CSA median value were significantly decreased, but in the CXCL14 plasmid-injected group, the CSA value and CSA median value were significantly increased, so that the overexpression of CXCL14 inhibited LPS-induced muscular atrophy.

In addition, as shown in FIG. 12, it was confirmed that the expression of CXCL14 increased the activity of FOXO1 and FOXO3, and decreased the expression of the muscle protein degradation enzymes Atrogin-1 and MuRF1.

### <Example 7> Confirmation of Inhibition of Dexamethasone-Induced Muscular Atrophy by Local Expression of CXCL14

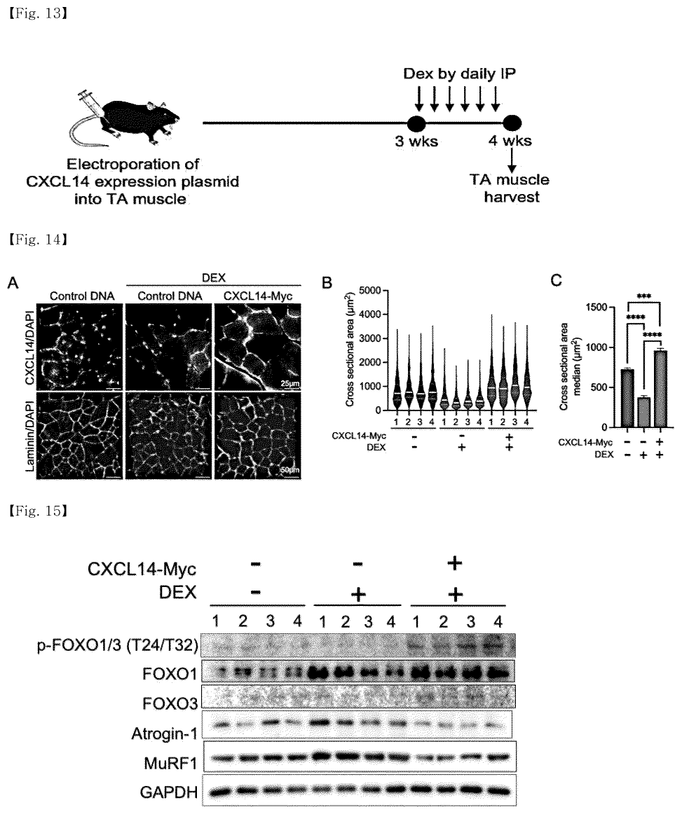

It was confirmed whether the local expression of CXCL14 of the present disclosure inhibited dexamethasone (DEX)-induced muscular atrophy in vivo. Specifically, a CXCL14 gene expression plasmid (20 μg) was injected into the TA muscle of C57BL/6 mice by electroporation, and then stabilized for 3 weeks. Thereafter, DEX (20 mg/kg of body weight) was injected intraperitoneally once a day for 6 days to induce muscular atrophy. One week after DEX injection, the mice were humanely sacrificed, TA muscles were extracted, and muscle transverse paraffin sections were prepared. The overall experimental process was shown in FIG. 13. Thereafter, the expression of CXCL14 protein and laminin protein was confirmed by immunofluorescence staining. In addition, the cross-sectional area (CSA) of a myofiber cell was measured using ImageJ program, which was used as a measure of myofiber cell mass.

In addition, Western blot analysis was used to measure the expression and activation levels of key factors in protein synthesis and degradation processes in muscle tissue lysates.

As a result, as shown in FIG. 14, it was confirmed that compared to the control group, in the DEX-injected group, the CSA value and CSA median value were significantly decreased, but in the CXCL14 plasmid-injected group, the CSA value and CSA median value were significantly increased, so that the overexpression of CXCL14 inhibited DEX-induced muscular atrophy.

In addition, as shown in FIG. 15, it was confirmed that the expression of CXCL14 increased the activity of FOXO1 and FOXO3, and decreased the expression of the muscle protein degradation enzymes Atrogin-1 and MuRF1.

Therefore, according to the present disclosure, it was confirmed that CXCL14 increased the muscle differentiation and synthesis, thereby increasing muscle mass. In addition, it was confirmed that in LPS-induced muscular atrophy or DEX-induced muscular dystrophy, CXCL14 inhibited muscle loss, increased the expression of muscle synthesis and differentiation factors, and inhibited the expression of muscle degradation proteins. In addition, it was confirmed that CXCL14 promoted the muscle synthesis and differentiation and inhibited the muscle degradation even in vivo.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method for preventing or treating muscle disease, the method comprising administering to a subject in need thereof a CXC motif chemokine ligand 14 (CXCL14) protein as an active ingredient.

2. The method of claim 1, wherein the CXCL14 protein comprises an amino acid sequence represented by SEQ ID NO: 1.

3. The method of claim 1, further comprising:
a CXCL14 expression vector.

4. The method of claim 3, wherein the CXCL14 expression vector is a plasmid comprising a base sequence represented by SEQ ID NO: 2 or 3.

5. The method of claim 1, wherein the CXCL14 increases muscle mass.

6. The method of claim 5, wherein the increasing of the muscle mass increases the number of myosin heavy chain positive cells.

7. The method of claim 1, wherein the CXCL14 increases the expression of muscle synthesis and differentiation factors.

8. The method of claim 7, wherein the muscle synthesis and differentiation factors are AKT (protein kinase B)-S6K (Ribosomal protein S6 kinase) pathway factors.

9. The method of claim 8, wherein the AKT-S6K pathway factor is selected from the group consisting of p-ATK (T308), p-AKT (S473), total AKT, p-S6K (T389), total S6K, p-mTOR, and total mTOR.

10. The method of claim 7, wherein the muscle synthesis and differentiation factors are Forkhead box protein 01 or Forkhead box protein 03 (FOXO3).

11. The method of claim 1, wherein the CXCL14 inhibits the expression of a muscle degradation factor.

12. The method of claim 11, wherein the muscle degradation factor is Atrogin-1 or Muscle RING-finger protein-1 (MuRF1).

13. The method of claim 1, wherein the muscle disease is disease selected from the group consisting of muscular atrophy, myopathy, muscular degeneration, myasthenia, muscular injury, dystrophinopathy, myopathy, muscular dystrophy, cachexia, and sarcopenia.

14. The method of claim 1, wherein the muscle disease is induced by bacterial infection or steroid.

15. A method for preventing or improving muscle disease, the method comprising administering to a subject in need thereof a CXC motif chemokine ligand 14 (CXCL14) protein as an active ingredient.


SEQUENCE LISTING (3 sequences)

SEQ ID NO: 1 - protein, 77 aa, synthetic construct
SKCKCSRKGPKIRYSDVKKLEMKPKYPHCEEKMVIITTKSVSRYRGQEHCLHPKLQSTKRFIKWYNAWNEKRRVYEE

SEQ ID NO: 2 - DNA, 5297 bp, synthetic construct
  source 1..5297: mol_type = other DNA
gacggatcgggagatctcccgatcccctatggtgcactctcagtacaatctgctctgatgaagccagtatctgctccctgcttgtgtgttggaggtcgctgagtagtgcgttaagctacaacaaggcaaggcttgaccgacaattgcatgaagaatctgcgcgttttgcgctgcttcgcgagtacatttatattggctcatgtccaatatttgacattgattattgactagttattaatagtaatcaattacggggtcatcccatatatggagttccgcgttacataacttacggtaaatggcccgcctgcaacgacccccgcccattgacgtcaataatgacgtatgttcccatagtaagactttccattgacgtcaatgggtggagtatttacggtaaactgcccacttcaagtgtatcatatgccaagtccgccccctattgacgtcaatgacggtactggcattatgcccagtacatgaccttacgggactttcctacttggcagtattagtcatcgctattaccatggtgatgcggttttggcagtacaccaatggcggtttgactcacggggatttccaagtctccaccccattgacgtcaatgttggcaccaaaatcaacgggactttccaaaatgtcgtaataaccccgcccaatgggcggtaggcgtgtacggtgggaggtctatataagcagagctcgtttcagatcctcactctcttccgcatcgctgtctgcgagggccagctgttgggaggacaaactcttcgcggtctttccagtactcttggatcggaaacccgtacggtactccgccaccgagggacctgagcgagtccgcatcgaccggatcgcgagaaaggcgtctaaccagtcacagtcgcaaggtaggctgagcaccgtggcgggtggcggtcggggttgtttctggcggaggtgctgctgatgatgtaacggtcttgagacggcggatggtcgaggtgaggtgtggcaggcttgagatcggtgagtactccctctcaaaagcgggcattacttctgcgctaagattgtcaacgaggaggatttgatattcacctggcccgatctggccatacacttgagatccactttgcctttctctccacaggtgtccactcccaggtccaagtttaaactttaatacgactcactataggggccgccaccatgccactgctgctcttgctgcctctgctttgggctggagctctggcttatccttacgacgtgcctgactacgcctccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaagagcagaaactcatctcagaagaggatctgtaagggcccgtttaaacccgctgatcagcctcgactgtgccttctagttgccagccatctgttgtttgcccctcccccgtgccttccttgaccctggacccactgtcctttcctaataaaatgaggaaattgcatcgcattgtctgagtctattctggggggtggggtggggcaggacagcaagggggaggattgggaaggcatgctggggatgcggtgggctctatggcttctgaggcggaaagaactctagggggtatccccacgcgccctgtagcggcgcattaagcgcggcgggacgcgcagcgtgaccgctacacttgccagcgccctagcgcccgctcctttccttcctttctcgccacgttcgcaggctttccccgtcaagctctaaatcgttagggttccgatttagtgctttacggcacctcgaccccaaaaaacttgaggttcacgtagtgggccatcgccctgatagacggtttttcgccctttgacacgttctttaatagtggactcttgttccaaactggaacaacactcaaccctattcttttgatttataagggattttgccgatttcggcctattggttaaaatttaacaaaaatttaacgcgaattaattctgtggaatgtgtgtcagttaagtccccaggctccccagcaggcagaagtatgcaaagcatgcatctcaatccaggtgtggaaagtccccaggctccccagcaggcagaagtatgcaaagcattagtcagcaaccatagtcccgcccctaactccgcccatcccgcccctagttccgcccattctccgccccatggctgactaattttttttatttatgcaccgcctctgcctctgagctattccagaagtagtgaggaggcttttttggatttgcaaaaagctctcgggagcttgtatatccattttcggatctgatcagaaaagcctgaactcaccgcgacgtctgtcgagaagtttctgatcgaaaagtctccgacctgatgcagctctcggagggcgaagaatctcgtgctttcagcgagggcgtggatatgtcctgcgggtaaatagctgcgccgatggtttctacatgtttatcggcactttgcatcggccgcgctcccgattccggaagtgcttaattcagcgagagcctgacctattgcatctcccgccgtgcacagggtgtcacctgcctgaaaccgaactgcccgctgttctgcagccggtcgcggaggcctcgctgcggccgatcttagccagacgagcgggttcggcccattcggaccggtcaatacactacatggcgtgatttcatatgcgcgattgctgatccccatggcaaactgtgatggacgacaccgtcagtgcgtccgtcgcgcaggctctctgctttgggccgaggactgccccgaagtccggcacctcgtgcacgcggatacaatgtcctgacggacaatggccgcataacagcggtcattgactggagctcggggattcccaatacgaggtcgccaacatcttcttctggaggccgtggtggagcagcagacgcgctacttcgagcggaggcatccggagcttgcaggatccgggcgtatatgctccgcattggtcttgaccaactctatcagagcttgatttcgatgatgcagcttgggcgcagggtcgatgcgacgcaatcgtccgaggactgtcgggcgtacacaaatcgcccgcagaagcgcggccgtctggacctagaagtactcgccgatagtggaaaccgacgccccagcactcgtccgaggagcacgtgctacgagatttcgattccaccgccgccttctatgaaaggttgtcgttttccgggacgctggctggatgatcctccagcgcggggatctcatgtcgcccaccccaacttgtttattgcagcttataatggttacaaataaagccaaatttcacaaataaagcatttttttcactgcattctagttgtggtttgtcaatgtatcttatcatgtctgtataccgtcgacctctagctagagcttgggtcatagctgtttcctgtgtgaaattgttatccgctcacaattccacacccggaagcataaagtgtaaagcctggggtgcctaatgagtgagctaactccgttgcgctcactgcccgctttccagtcgggaaacctgtcgtgccagctgtcggccaacgcgcggggagaggcggtttgcgtattgggcgctcttccgctctgactcgctgcgctcggtcgttcggctgcggcgagcggtatcagctcactaatacggttatccacagaatcaggggataacgcaggaaagaacatgtgaagcaaaaggccaggaaccgtaaaaaggccgcgttgctggcgtttttccatcccctgacgagcatcacaaaaatcgacgctcaagtcagaggtggcgaaactataaagataccaggcgtttccccctggaagctccctcgtgcgctctccttgccgcttaccggatacctgtccgcctttctcccttcgggaagcgtggcggctcacgctgtaggtatctcagttcggtgtaggtcgttcgctccaagctgacgaaccccccgttcagcccgaccgctgcgccttatccggtaactatcgtacccggtaagacacgacttatcgccactggcagcagccactggtaacaggcgaggtatgtaggcggtgctacagagttcttgaagtggtggcctaactacgaagaacagtatttggtatctgcgctctgctgaagccagttaccttcggagtagctcttgatccggcaaacaaaccaccgctggtagcggtggtttttttagcagattacgcgcagaaaaaaaggatctcaagaagatcctttgatctttctgagcgcggaacccctatttgtttatttttctaaatacattcaaatatgtgaattaattcttagaaaaactcatcgagcatcaaatgaaactgcaatttggattatcaataccatatttttgaaaaagccgtttctgtaatgaaggagaaggcagttccataggatggcaagatcctggtatcggtctgcgattccgactcaatacaacctattaatttcccctcgtcaaaaataaggttatcaagtgatgagtgacgactgaatccggtgagaatggcaaaagtttatgcatttcttttcaacaggccagccattacgctcgtcatcaaaatcactcgcatcaaccaaattcgtgattgcgcctgagcgagacgaaatacgcgatcgctgttaaaaggacaggaatcgaatgcaaccggcgcaggaacactgccagcgcatcaacaatgaatcaggatattcttctaatacctggaatgctgttttcccagggatcgcaaccatgcatcatcaggagtacggataaaatgcttgatggtcggaagaggcataaattccgtcagccagtttagtctgaccatctcatctgtaacatcattggcaacgcttgtttcagaaacaactctggcgcatcgggcttcccatacaatcgatagatgattgcccgacattatcgcgagcccatttatacccatataaatcagcatctttaatcgcggcctagagcaagacgtttcccgttgaatatggctcataacttactgtttatgtaagcagacagttttattgttcatgaccaaaatcccttttcgttccactgagcgtcagaccccgtagaaatccgcgcacatttccccgcctgacgtc

SEQ ID NO: 3 - DNA, 5324 bp, synthetic construct
  source 1..5324: mol_type = other DNA
gacggatcgggagatctcccgatcccctatggtgcactctcagtacaatctgctctgatgaagccagtatctgctccctgcttgtgtgttggaggtcgctgagtagtgcgttaagctacaacaaggcaaggcttgaccgacaattgcatgaagaatctgcgcgttttgcgctgcttcgcgagtacatttatattggctcatgtccaatatttgacattgattattgactagttattaatagtaatcaattacggggtcatcccatatatggagttccgcgttacataacttacggtaaatggcccgcctgcaacgacccccgcccattgacgtcaataatgacgtatgttcccatagtaagactttccattgacgtcaatgggtggagtatttacggtaaactgcccacttcaagtgtatcatatgccaagtccgccccctattgacgtcaatgacggtactggcattatgcccagtacatgaccttacgggactttcctacttggcagtattagtcatcgctattaccatggtgatgcggttttggcagtacaccaatggcggtttgactcacggggatttccaagtctccaccccattgacgtcaatgttggcaccaaaatcaacgggactttccaaaatgtcgtaataaccccgcccaatgggcggtaggcgtgtacggtgggaggtctatataagcagagctcgtttcagatcctcactctcttccgcatcgctgtctgcgagggccagctgttgggaggacaaactcttcgcggtctttccagtactcttggatcggaaacccgtacggtactccgccaccgagggacctgagcgagtccgcatcgaccggatcgcgagaaaggcgtctaaccagtcacagtcgcaaggtaggctgagcaccgtggcgggtggcggtcggggttgtttctggcggaggtgctgctgatgatgtaacggtcttgagacggcggatggtcgaggtgaggtgtggcaggcttgagatcggtgagtactccctctcaaaagcgggcattacttctgcgctaagattgtcaacgaggaggatttgatattcacctggcccgatctggccatacacttgagatccactttgcctttctctccacaggtgtccactcccaggtccaagtttaaactttaatacgactcactataggggccgccaccatgtccctgctcccacgccgcgcccctccggtcagcatgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaagagcagaaactcatctcagaagaggatctgtaagggcccgtttaaacccgctgatcagcctcgactgtgccttctagttgccagccatctgttgtttgcccctcccccgtgccttccttgaccctggacccactgtcctttcctaataaaatgaggaaattgcatcgcattgtctgagtctattctggggggtggggtggggcaggacagcaagggggaggattgggaaggcatgctggggatgcggtgggctctatggcttctgaggcggaaagaactctagggggtatccccacgcgccctgtagcggcgcattaagcgcggcgggacgcgcagcgtgaccgctacacttgccagcgccctagcgcccgctcctttccttcctttctcgccacgttcgcaggctttccccgtcaagctctaaatcgttagggttccgatttagtgctttacggcacctcgaccccaaaaaacttgaggttcacgtagtgggccatcgccctgatagacggtttttcgccctttgacacgttctttaatagtggactcttgttccaaactggaacaacactcaaccctattcttttgatttataagggattttgccgatttcggcctattggttaaaatttaacaaaaatttaacgcgaattaattctgtggaatgtgtgtcagttaagtccccaggctccccagcaggcagaagtatgcaaagcatgcatctcaatccaggtgtggaaagtccccaggctccccagcaggcagaagtatgcaaagcattagtcagcaaccatagtcccgcccctaactccgcccatcccgcccctagttccgcccattctccgccccatggctgactaattttttttatttatgcaccgcctctgcctctgagctattccagaagtagtgaggaggcttttttggatttgcaaaaagctctcgggagcttgtatatccattttcggatctgatcagaaaagcctgaactcaccgcgacgtctgtcgagaagtttctgatcgaaaagtctccgacctgatgcagctctcggagggcgaagaatctcgtgctttcagcgagggcgtggatatgtcctgcgggtaaatagctgcgccgatggtttctacatgtttatcggcactttgcatcggccgcgctcccgattccggaagtgcttaattcagcgagagcctgacctattgcatctcccgccgtgcacagggtgtcacctgcctgaaaccgaactgcccgctgttctgcagccggtcgcggaggcctcgctgcggccgatcttagccagacgagcgggttcggcccattcggaccggtcaatacactacatggcgtgatttcatatgcgcgattgctgatccccatggcaaactgtgatggacgacaccgtcagtgcgtccgtcgcgcaggctctctgctttgggccgaggactgccccgaagtccggcacctcgtgcacgcggatacaatgtcctgacggacaatggccgcataacagcggtcattgactggagctcggggattcccaatacgaggtcgccaacatcttcttctggaggccgtggtggagcagcagacgcgctacttcgagcggaggcatccggagcttgcaggatccgggcgtatatgctccgcattggtcttgaccaactctatcagagcttgatttcgatgatgcagcttgggcgcagggtcgatgcgacgcaatcgtccgaggactgtcgggcgtacacaaatcgcccgcagaagcgcggccgtctggacctagaagtactcgccgatagtggaaaccgacgccccagcactcgtccgaggagcacgtgctacgagatttcgattccaccgccgccttctatgaaaggttgtcgttttccgggacgctggctggatgatcctccagcgcggggatctcatgtcgcccaccccaacttgtttattgcagcttataatggttacaaataaagccaaatttcacaaataaagcatttttttcactgcattctagttgtggtttgtcaatgtatcttatcatgtctgtataccgtcgacctctagctagagcttgggtcatagctgtttcctgtgtgaaattgttatccgctcacaattccacacccggaagcataaagtgtaaagcctggggtgcctaatgagtgagctaactccgttgcgctcactgcccgctttccagtcgggaaacctgtcgtgccagctgtcggccaacgcgcggggagaggcggtttgcgtattgggcgctcttccgctctgactcgctgcgctcggtcgttcggctgcggcgagcggtatcagctcactaatacggttatccacagaatcaggggataacgcaggaaagaacatgtgaagcaaaaggccaggaaccgtaaaaaggccgcgttgctggcgtttttccatcccctgacgagcatcacaaaaatcgacgctcaagtcagaggtggcgaaactataaagataccaggcgtttccccctggaagctccctcgtgcgctctccttgccgcttaccggatacctgtccgcctttctcccttcgggaagcgtggcggctcacgctgtaggtatctcagttcggtgtaggtcgttcgctccaagctgacgaaccccccgttcagcccgaccgctgcgccttatccggtaactatcgtacccggtaagacacgacttatcgccactggcagcagccactggtaacaggcgaggtatgtaggcggtgctacagagttcttgaagtggtggcctaactacgaagaacagtatttggtatctgcgctctgctgaagccagttaccttcggagtagctcttgatccggcaaacaaaccaccgctggtagcggtggtttttttagcagattacgcgcagaaaaaaaggatctcaagaagatcctttgatctttctgagcgcggaacccctatttgtttatttttctaaatacattcaaatatgtgaattaattcttagaaaaactcatcgagcatcaaatgaaactgcaatttggattatcaataccatatttttgaaaaagccgtttctgtaatgaaggagaaggcagttccataggatggcaagatcctggtatcggtctgcgattccgactcaatacaacctattaatttcccctcgtcaaaaataaggttatcaagtgatgagtgacgactgaatccggtgagaatggcaaaagtttatgcatttcttttcaacaggccagccattacgctcgtcatcaaaatcactcgcatcaaccaaattcgtgattgcgcctgagcgagacgaaatacgcgatcgctgttaaaaggacaggaatcgaatgcaaccggcgcaggaacactgccagcgcatcaacaatgaatcaggatattcttctaatacctggaatgctgttttcccagggatcgcaaccatgcatcatcaggagtacggataaaatgcttgatggtcggaagaggcataaattccgtcagccagtttagtctgaccatctcatctgtaacatcattggcaacgcttgtttcagaaacaactctggcgcatcgggcttcccatacaatcgatagatgattgcccgacattatcgcgagcccatttatacccatataaatcagcatctttaatcgcggcctagagcaagacgtttcccgttgaatatggctcataacttactgtttatgtaagcagacagttttattgttcatgaccaaaatcccttttcgttccactgagcgtcagaccccgtagaaatccgcgcacatttccccgcctgacgtc